(19) United States
(12) Patent Application Publication
(10) Pub. No.: US 2024/0301497 A1
(43) Pub. Date: Sep. 12, 2024

(54) METHODS FOR DIAGNOSING NASAL INTESTINAL TYPE ADENOCARCINOMAS

(71) Applicants: INSERM (INSTITUT NATIONAL DE LA SANTÉ ET DE LA RECHERCHE MÉDICALE), Paris (FR); UNIVERSITÉ DE LORRAINE, Nancy (FR); CENTRE HOSPITALIER ET UNIVERSITAIRE DE NANCY (CHU), Nancy (FR)

(72) Inventors: Rémi HOULGATTE, Vandoeuvre-lès-Nancy (FR); Abderrahim OUSSALAH, Vandoeuvre-lès-Nancy (FR); Jean-Louis GUEANT, Vandoeuvre les Nancy (FR); Patrice GALLET, Vandoeuvre les Nancy (FR); Roger JANKOWSKI, Vandoeuvre les Nancy (FR)

(21) Appl. No.: 17/769,474

(22) PCT Filed: Oct. 16, 2020
(86) PCT No.: PCT/EP2020/079230

§ 371 Date: Apr. 15, 2022

(30) Foreign Application Priority Data
Oct. 17, 2019 (EP) 19306355.9

(51) Int. Cl. C12Q 1/6886; A61B 10/02

(52) U.S. Cl. CPC C12Q 1/6886; A61B 10/02; A61B 2010/0216; C12Q 2600/154; C12Q 2600/158

(57) ABSTRACT

The present invention relates to methods and compositions for the diagnostic and for the treatment of nasal intestinal type adenocarcinomas (ITAC). The inventors used a non-invasive brushing technique, which permits to identify transcriptomic and methylation modifications that are consistent with phenotypic profiles and ITACs natural history. Thus, they identified CACNA1C as a new predictive marker of ITAC. In particular, the invention relates to a method of determining whether a subject has or is at risk of having nasal intestinal type adenocarcinoma (ITAC) comprising determining the expression level of CACNA1C in a sample wherein said expression level indicates whether the subject has or is at risk of having a nasal intestinal type adenocarcinoma (ITAC).

### FIELD OF THE INVENTION

The invention is in the field of oncology. More particularly, the present invention also relates to methods and compositions for the diagnostic and for the treatment of nasal intestinal type adenocarcinomas.

### BACKGROUND OF THE INVENTION

Among sinonasal tumors, intestinal type adenocarcinomas (ITACs) constitute a very particular subgroup. These tumors are indeed typically induced by chronic wood dust exposure, which might be considered as a sine qua non condition [De Gabory 2010; Gallet 2018]. The risk of developing an ITAC increases with exposure duration (increased after 1 year), exposure intensity (increased above 1 mg dust/m3) and latency (after 20 years) [Demers, 1995]. While a short exposure (<3 years) might be sufficient, there is a very significant increase in risk with duration of exposure (OR=5.3 for less than 5 years of exposure, 10.7 for 10-19 years and 36.7 for more than 30 years) [Carton, 2002]. ITACs development is usually delayed and average latency (mean time to onset of the disease after first exposure to wood dust) is long (28-40 years [Choussy, 2008; Demers, 1995], so that 90% of patients are over 50 years of age [De Gabory, 2010; Gallet, 2018]. A huge majority of woodworkers are male, so that ITACs affect men in almost all cases.

It has been demonstrated that ITACs arise from the olfactory cleft, which is the confluence of two tissues of different embryological origin: respiratory epithelium, and olfactory epithelium [Jankowski, 2007; Georgel, 2009]. For some authors, ITACs might be preceded by a phase of metaplasia [Bonato, 1989; Choi, 2003; Donhuijsen, 2004; Kennedy, 2004; Vivanco, 2011; Wilhelmsson, 1984]. The transformation of the normal epithelium into an ITAC relies on a key event, the overexpression of CDX2 [Kennedy, 2004; De Gabory, 2010]. This phenomenon is constant, as CDX2 expression seems mandatory for the intestinal organization of these tumors [De Gabory, 2010]. The other genomic abnormalities observed (such as p53 mutations, loss of heterozygoties . . . ) are more inconstant, and associated with more advanced forms of adenocarcinomas, thus suggesting that they could be late events in the natural history of adenocarcinomas [De Gabory, 2010; Gallet, 2018]. The precise role of CDX2 in carcinogenesis is, however, not precisely established: chronic inflammation might trigger CDX2 expression thus leading to metaplasia. But this has never been confirmed.

Early diagnosis would offer a significant benefit: the 5-year disease free survival is 100% for T1 tumors, 85-100% for T2, and <40% for T4 [Choussy, 2008]. Unfortunately the diagnosis is usually delayed because symptoms are not very marked and not specific. Organized screening of exposed woodworkers is therefore likely to enable early diagnosis (metaplasia or small tumors), but screening is currently relies only on endoscopy and the olfactory cleft is a narrow space, sometimes difficult to access and explore [Porez, 2011]. Biopsies may be very difficult to perform in consultation given the anatomical conditions (narrowness of the olfactory cleft, septal deviation, polyps . . . ).

It would therefore be important to dispose from an alternative screening method. But there is to date no study on transcriptomic and methylation abnormalities in woodworker's nasal epithelium. Thus the inventors' objectives were to investigate transcriptomic and methylation differences between healthy non-exposed and tumoral olfactory cleft mucosa and to compare transcriptomic profiles between non-exposed, wood dust exposed and ITAC mucosa cells. Surprisingly the inventors identified CACNA1C as a new predictive marker of ITAC.

### SUMMARY OF THE INVENTION

The present invention also relates to methods and compositions for the diagnostic and for the treatment of nasal intestinal type adenocarcinomas. The present invention relates to the use of CACNA1C as biomarkers of nasal intestinal type adenocarcinoma. In particular, the invention is defined by the claims.

### DETAILED DESCRIPTION OF THE INVENTION

Nasal intestinal type adenocarcinomas (ITAC) are strongly related to chronic wood dust exposure: the intestinal phenotype rely on CDX2 overexpression but underlying molecular mechanisms remain unknown.

The inventors proposed an original approach to investigate wood dust induced modifications: They developed a non-invasive sampling method to take samples from the olfactory cleft in normal volunteers (non-exposed), exposed woodworkers, and exposed woodworkers with a tumor, with the aim to investigate wood dust induced transcriptomic and methylation modifications. The first objective of the study was to identify the main transcriptomic and methylation modifications in olfactory cleft mucosa between woodworkers presenting a tumor and healthy controls. The second objective of the study was to identify the main transcriptomic modifications between tumoral cells, wood dust exposed non-tumoral mucosa and non-exposed mucosa. Overall, the work of the inventors might contribute to ITACs carcinogenesis understanding and help in defining a new screening method.

Thus the first object of the present invention relates to a method of determining whether a subject has or is at risk of having nasal intestinal type adenocarcinoma (ITAC) comprising determining the expression level of CACNA1C in a sample wherein said expression level indicates whether the subject has or is at risk of having a nasal intestinal type adenocarcinoma (ITAC).

As used herein, the term “subject” denotes a mammal, such as a rodent, a feline, a canine, and a primate. Particularly, the subject according to the invention is a human. As used herein, the term “subject” encompasses “patient”.

In some embodiment, the subject of the invention is a person who is in contact with dust, wood dust, mineral dust (e.g. magnesia, chalk) or coal. For example the subject is a woodworker, a miner, a climber.

As used herein the term “woodworker” refers to a worker in wood, as a carpenter, joiner, or cabinetmaker.

As used herein, the term “wood” has its general meaning in the art and refers to the hard fibrous material that forms the main substance of the trunk or branches of a tree or shrub, used for fuel or timber. Examples of common wood known in the art include, but are not limited to, pine, spruce, larch, juniper, aspen, hornbeam, birch, alder, beech, oak, elm, cherry, pear, maple, linden, ash.

In some embodiment, the wood can be find is in different form. Examples of wood form known in the art include, but are not limited to dust, soot, small wood, branches, chips, sawdust, wood flour and sawmill waste resulting from lumber processing. Particularly, the wood according to the invention is in the form of dust.

As used herein, the term “healthy” has its general meaning in the art and refers to a subject in a good physical and mental condition, in good health.

As used herein the term “healthy non-exposed” refers to a healthy subject non-exposed to wood dust. The term “healthy non-exposed” is also defined by “N”

As used herein the term “exposed without tumor” refers to a subject, in particular woodwokers, exposed to wood dust who does not have a tumor. The term “exposed without tumor” “is also defined by “E”

As used herein the term “exposed with tumor” refers to a subject exposed to wood dust who has ITAC. The term “exposed with tumor” is also defined by “T”

As used herein, the term “C” refers to the contralateral side of woodworker with tumor.

As used herein, the term “sample” refers to any sample obtained from a subject, such as a serum sample, a plasma sample, a urine sample, a blood sample, a lymph sample, or a tissue sample. In a particular embodiment, the sample is a tissue sample.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

Thus the expression “determining whether a patient is at risk of having an ITAC” as used herein means that the patient to be analyzed by the method of the present invention is allocated either into the group of patients of a population having an elevated risk, or into a group having a reduced risk of having an ITAC. An elevated risk as referred to in accordance with the present invention, preferably, means that the risk of having an ITAC within a predetermined predictive window is elevated significantly (i.e. increased significantly) for a patient with respect to the average risk measured in a general population. A reduced risk as referred to in accordance with the present invention, preferably, means that the risk of having an ITAC within a predetermined predictive window is reduced significantly for a patient with respect to the average risk measured in the general population. Particularly, a significant increase or reduction of a risk is an increase or reduction or a risk of a size which is considered to be significant for prognosis, particularly said increase or reduction is considered statistically significant. The terms “significant” and “statistically significant” are known by the person skilled in the art. Thus, whether an increase or reduction of a risk is significant or statistically significant can be determined without further ado by the person skilled in the art using various well known statistic evaluation tools.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

In a particular embodiment, the sample is collected in olfactory clefts.

The inventors developed a non-invasive sampling method to take samples from the olfactory cleft of healthy non-exposed subject, exposed without tumor subject and exposed with tumor subject. In some embodiment, the non-invasive sampling method is a brushing technique comprising the insertion of a brush into the nasal cavity.

In some embodiments, when the subject suffers from a cancer, the tissue sample is a tumor tissue sample. As used herein, the term “tumor tissue sample” means any tissue tumor sample derived from the subject. Said tissue sample is obtained for the purpose of the in vitro evaluation. In some embodiments, the tumor sample may result from the tumor resected from the subject. In some embodiments, the tumor sample may result from a biopsy performed in the primary tumour of the subject or performed in metastatic sample distant from the primary tumor of the subject. In some embodiments, the tumor tissue sample encompasses a global primary tumor (as a whole), a tissue sample from the center of the tumor, a tumor tissue sample collected prior surgery (for follow-up of subjects after treatment for example), and a distant metastasis. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded).

As used herein, the term “nasal intestinal type adenocarcinoma” (ITAC) has its general meaning in the art and refers to an epithelial, nonsquamous cell tumor of the sinonasal tract. Based on histopathologic parameters, ITACs are into five categories: papillary, colonic, solid, mucinous, and mixed subtypes (Barnes L., 1986).

The inventors have showed that woodworkers with ITAC exhibited an overexpression of the CACNA1C gene.

As used herein, the term “gene” has its general meaning in the art and refers a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

As used herein, the term “CACNA1C” or “calcium voltage-gated channel subunit alpha1 C” has its general meaning in the art and relates to an alpha-1 subunit of a voltage-dependent calcium channel. The naturally occurring human CACNA1C gene has a nucleotide sequence having the following number: Gene ID: 775 and the naturally occurring human CACNA1C protein has an aminoacid sequence as shown in Genbank Accession numbers: AAI46847.1

As used herein, the term “CACNA1C-AS1” or “CACNA1C antisense RNA 1” has its general meaning in the art and relates a RNA Gene. The naturally occurring human CACNA1C-AS1 has a nucleotide sequence having the following number: Gene ID: 100652846.

As used herein, the term “SLC26A10” or “Solute Carrier Family 26 Member 10”. The naturally occurring human SLC26A10 gene has a nucleotide sequence having the following number: Gene ID: 65012 and the naturally occurring human SLC26A10 protein has an aminoacid sequence as shown in Genbank Accession numbers: NP_597996.2.

In the present specification, the name of each of the genes of interest refers to the internationally recognised name of the corresponding gene, as found in internationally recognised gene sequences and protein sequences databases, in particular in the database from the HUGO Gene Nomenclature Committee, that is available notably at the following Internet address: https://www.genenames.org/. In the present specification, the name of each of the various biological markers of interest may also refer to the internationally recognised name of the corresponding gene, as found in the internationally recognised gene sequences and protein sequences databases ENTREZ ID, Genbank, TrEMBL or ENSEMBL. Through these internationally recognised sequence databases, the nucleic acid sequences corresponding to each of the gene of interest described herein may be retrieved by the one skilled in the art. (see Table A).

TABLE A
the genes names of the present invention

| Name | Symbol | ENTREZ ID | Regulation |
|---|---|---|---|
| calcium voltage-gated channel subunit | CACNA1C | 775 | up |
| alpha1 C | | | |
| CACNA1C Intronic Transcript 3 | CACNA1C-IT3 | 100874370 | up |
| N-Myc Downstream Regulated 1 | NDRG1 | 10397 | up |
| hemoglobin subunit alpha 2 | HBA2 | 3040 | up |
| Hemoglobin subunit beta | HBB | 3043 | up |
| Immunoglobulin lambda-like | IGLL5 | 100423062 | up |
| polypeptide 5 | | | |
| Calmodulin-3 | CALM3 | 808 | up |
| protein tyrosine phosphatase 4A3 | PTP4A3 | 11156 | up |
| BTG Anti-Proliferation Factor 2 | BTG2 | 7832 | up |
| PAXX Non-Homologous End Joining | PAXX | 286257 | up |
| Factor | | | |
| KIAA0040 | KIAA0040 | 9674 | up |
| FLOT1 | FLOT1 | 10211 | up |
| Dipeptidyl peptidase 7 | DPP7 | 29952 | up |
| Hemoglobin Subunit Alpha 1 | HBA1 | 3039 | up |
| Hydroxypyruvate Isomerase | HYI | 81888 | up |
| Vimentin | VIM | 7431 | up |
| GNAS complex locus | GNAS | 2778 | up |
| Small subunit processome component 20 | UTP20 | 27340 | down |
| homolog | | | |
| Nucleoporin 93 | NUP93 | 9688 | down |
| Glutamate Ionotropic Receptor Kainate | GRIK2 | 2898 | down |
| Type Subunit 2 | | | |
| Protein Phosphatase 3 Catalytic Subunit | PPP3CC | 5533 | down |
| Gamma | | | |
| Leucine Rich Repeats And | LRIG3 | 121227 | down |
| Immunoglobulin Like Domains 3 | | | |
| CUB And Sushi Multiple Domains 1 | CSMD1 | 64478 | down |
| ATPase Copper Transporting Beta | ATP7B | 540 | down |
| Eukaryotic Translation Initiation Factor | EIF2AK3 | 9451 | down |
| 2 Alpha Kinase 3 | | | |
| NIPA Like Domain Containing 2 | NIPAL2 | 79815 | down |
| Carnitine O-Octanoyltransferase | CROT | 54677 | down |
| NIPA Like Domain Containing 3 | NIPAL3 | 57185 | down |
| Chaperonin Containing TCP1 Subunit | CCT6B | 10693 | down |
| 6B | | | |
| ALG9 Alpha-1,2-Mannosyltransferase | ALG9 | 79796 | down |
| Transmembrane And Tetratricopeptide | TMTC3 | 160418 | down |
| Repeat Containing 3 | | | |
| Thyroid Hormone Receptor Interactor 12 | TRIP12 | 9320 | down |

In some embodiment, the expression level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 and/or 31, genes is determined.

In some embodiment, the expression level of CACNA1C gene is determined.

In some embodiment, the expression level of CACNA1C-IT3 gene is determined.

In some embodiment, the method of the present invention further comprises determining the expression level of at least one gene selected from the group consisting of NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3, or TRIP12.

In some embodiment, the method of the present invention further comprises determining the expression level of the genes consisting of NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and TRIP12.

In some embodiment, the method of the present invention further comprises determining the expression level of CACNA1C in combination with at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3, or TRIP12.

In some embodiment, the method of the present invention further comprises determining the expression level of CACNA1C in combination with the genes consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and TRIP12.

In some embodiment, the expression level of NDRG1 gene is determined.

In some embodiment, the expression level of HBA2 gene is determined.

In some embodiment, the expression level of HBB gene is determined.

In some embodiment, the expression level of IGLL5 gene is determined.

In some embodiment, the expression level of CALM3 gene is determined.

In some embodiment, the expression level of PTP4A3 gene is determined.

In some embodiment, the expression level of BTG2 gene is determined.

In some embodiment, the expression level of PAXX gene is determined.

In some embodiment, the expression level of KIAA0040 gene is determined.

In some embodiment, the expression level of FLOT1 gene is determined.

In some embodiment, the expression level of DPP7 gene is determined.

In some embodiment, the expression level of HBA1 gene is determined.

In some embodiment, the expression level of HYI gene is determined.

In some embodiment, the expression level of VIM gene is determined.

In some embodiment, the expression level of GNAS gene is determined.

In some embodiment, the expression level of UTP20 gene is determined.

In some embodiment, the expression level of NUP93 gene is determined.

In some embodiment, the expression level of GRIK2 gene is determined.

In some embodiment, the expression level of PPP3CC gene is determined.

In some embodiment, the expression level of LRIG3 gene is determined.

In some embodiment, the expression level of CSMD1 gene is determined.

In some embodiment, the expression level of ATP7B gene is determined.

In some embodiment, the expression level of EIF2AK3 gene is determined.

In some embodiment, the expression level of NIPAL2 gene is determined.

In some embodiment, the expression level of CROT gene is determined.

In some embodiment, the expression level of NIPAL3 gene is determined.

In some embodiment, the expression level of CCT6B gene is determined.

In some embodiment, the expression level of ALG9 gene is determined.

In some embodiment, the expression level of TMTC3 gene is determined.

In some embodiment, the expression level of TRIP12 gene is determined.

The inventors also identified, in methylome analysis, CACNA1C-AS1 as a site of interest. This locus corresponds to the CpG island “CpG: 84” which is located in the promotor 5′ region of the CACNA1C-AS1 and the 3′ UTR region of the CACNA1C gene. The CpG probes reported in this locus exhibited a hemi-methylated profile among cancerous sample.

As used herein the term “methylation” has its general meaning in this art and refers to the addition of a methyl group on a substrate, or the substitution of an atom (or group) by a methyl group. Methylation is a form of alkylation, with a methyl group, rather than a larger carbon chain, replacing a hydrogen atom. Methylation is accomplished by enzymes; methylation can modify heavy metals, regulate gene expression, RNA processing and protein function. It has been recognized as a key process underlying epigenetics.

As used herein, the term “methylation level” refers to the methylation level of CACNA1C-AS1.

Typically, the methylation level of the gene CACNA1C-AS1 may be determined by any technology known by a person skilled in the art. In particular, each gene methylation level may be measured at the genomic and/or nucleic and/or protein level. In a particular embodiment, the methylation level of gene is determined by measuring the amount of nucleic acid transcripts of each gene. In another embodiment, the methylation level is determined by measuring the amount of each gene corresponding protein. The amount of nucleic acid transcripts can be measured by any technology known by a man skilled in the art.

Methylation level of a gene may be expressed as absolute level or normalized level. Typically, levels are normalized by correcting the absolute level of a gene by comparing its methylation level to the methylation level of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the level in one sample, e.g., a subject sample, to another sample, or between samples from different sources.

In some embodiment, the method of the present invention further comprises determining the expression level of CACNA1C in combination with at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3, or TRIP12 and/or the methylation level of CACNA1C-AS1.

In some embodiment, the method of the present invention further comprises determining the methylation expression level of CACNA1C in combination with the genes consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and TRIP12 and/or the methylation level of CACNA1C-AS1.

In some embodiments, the expression level of the gene and/or the methylation level of the gene is determined at nucleic acid level. Typically, the measure may be carried out directly on an extracted messenger RNA (mRNA) sample, or on retrotranscribed complementary DNA (cDNA) prepared from extracted mRNA by technologies well-known in the art. Typically, the level of a gene is determined by determining the quantity of mRNA. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3 (4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dichlorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes. Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase. Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .l. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the expression level of the gene and/or the methylation level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-overlapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tissue sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

In some embodiments, when multi-quantification is required, use of beads bearing binding partners of interest may be preferred. In some embodiments, the bead may be a cytometric bead for use in flow cytometry. Such beads may for example correspond to BD™ Cytometric Beads commercialized by BD Biosciences (San Jose, California). Typically cytometric beads may be suitable for preparing a multiplexed bead assay. A multiplexed bead assay, such as, for example, the BDm Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BDm Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In some embodiments, the expression level of the gene and/or the methylation level of the gene are determined by immunohistochemistry (IHC). Immunohistochemistry typically includes the following steps i) fixing said tissue sample with formalin, ii) embedding said tissue sample in paraffin, iii) cutting said tissue sample into sections for staining, iv) incubating said sections with the binding partner specific for the marker, v) rinsing said sections, vi) incubating said section with a biotinylated secondary antibody and vii) revealing the antigen-antibody complex with avidin-biotin-peroxidase complex. Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 1251) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

Multiplex tissue analysis techniques are particularly useful for quantifying several markers in the tissue sample. Such techniques should permit at least five, or at least ten or more biomarkers to be measured from a single tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, MD).

In some embodiments, the L-IHC method can be performed on any of a variety of tissue samples, whether fresh or preserved. The samples included core needle biopsies that were routinely fixed in 10% normal buffered formalin and processed in the pathology department. Standard five μιη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μιη thick coated polymer backbone with 0.4μιη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, MD). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, MD). That technique is described in U.S. Pat. No. 7,473,532.

In some embodiments, the method of the present invention utilized the multiplex IHC technique developed by GE Global Research (Niskayuna, NY). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal.

Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the subject, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the method of the present invention further comprises comparing the expression level of the genes of the invention with a predetermined reference value wherein detecting a difference between the expression level of the genes of the invention and the predetermined reference value indicates whether the subject is or is not at risk of having a ITAC.

In some embodiments, the method of the present invention further comprises comparing the methylation level of the genes of the invention with a predetermined reference value wherein detecting a difference between the methylation level of the genes of the invention and the predetermined reference value indicates whether the subject is or is not at risk of having a ITAC.

In some embodiments, the predetermined reference value is a relative to a number or value derived from population studies, including without limitation, subjects of the same or similar age range, subjects in the same or similar ethnic group, and subjects having the same severity of lesion. Such predetermined reference values can be derived from statistical analyses and/or risk prediction data of populations obtained from mathematical algorithms and computed indices. In some embodiments, retrospective measurement of the level of the marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, the term “up” in Table A indicates that the expression level of the gene is significantly higher than the expression level of the same gene in a population of healthy subjects. Conversely, the term “down” in Table A indicates that the expression level of the gene is significantly lower than the expression level of the same gene in a population of healthy subjects.

In some embodiment, when the expression level of the gene of CACNAC1 is higher than the same gene in a population of healthy subjects, it is considered that the subject has or is at a risk of having ITAC.

In some embodiment, when the expression level of the genes of CACNA1C-IT3, NDRG1, HBA2, HBB, IGLL5, ALM3, PTP4A3, BTG2, PAXX, KIAA0040, FLOT1, DPP7, HBA1, HYI, VIM, GNAS is higher than the same genes in a population of healthy subjects, it is considered that the subject has or is at a risk of having ITAC.

In some embodiment, when the expression level of the genes of UTP20, NUP93, GRIK2, PPP3CC, LRIG3, CSMD1, ATP7B, EIF2AK3, NIPAL2, CROT, NIPAL3, CCT6B, ALG9, TMTC3, TRIP12 is lower than the same genes in a population of healthy subjects, it is considered that the subject has or is at a risk of having ITAC.

In some embodiment, when the methylation level of CACNA1C-AS1 exhibited a hemi-methylated profile, it is considered that the subject has or is at a risk of having ITAC.

In some embodiments, a score which is a composite of several different genes is determined and compared to the predetermined reference value wherein a difference between said score and said predetermined reference value is indicative whether the subject has or is at risk of having a ITAC.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF). In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. A SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of a plurality of markers in the sample; b) implementing a classification algorithm on data comprising the quantified plurality of markers so as to obtain an algorithm output; c) determining the probability that the subject will develop a cancer from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

### Method for Treating Cancer

Accordingly, the method of the present invention is also suitable for determining whether a subject suffering from ITAC is eligible for a treatment or even for surgery. The method of the present invention offers the possibility of performing less invasive surgeries (e.g. preservation of the eye, no opening of the base of the skull).

Typically, the subject is treating with chemotherapy, radiotherapy, and immunotherapy.

The invention relates to a method for treating a subject suffering from ITAC, wherein said method comprises the step of:
(i) Determining in a biological sample obtained from said subject the expression level of CACNA1C
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent.

The invention relates to a method for treating a subject suffering from ITAC, wherein said method comprises the step of:
(i) Determining in a biological sample obtained from said subject the expression level of CACNA1C gene in combination with at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and/or TRIP12.
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent.

The invention relates to a method for treating a subject suffering from ITAC, wherein said method comprises the step of:
(i) Determining in a biological sample obtained from said subject the expression level of CACNA1C gene in combination with the genes consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and/or TRIP12.
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent.

The invention relates to a method for treating a subject suffering from ITAC, wherein said method comprises the step of:
(i) Determining in a biological sample obtained from said subject the methylation level of CACNA1C-AS1
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent.

The invention relates to a method for treating a subject suffering from ITAC, wherein said method comprises the step of:
(i) Determining in a biological sample obtained from said subject the expression level of CACNA1C gene in combination with at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and/or TRIP12 and/or the methylation level of CACNA1C-AS1.
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent.

The invention relates to a method for treating a subject suffering from ITAC, wherein said method comprises the step of:
(i) Determining in a biological sample obtained from said subject the expression level of CACNA1C gene in combination with the genes consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and/or TRIP12.
(ii) Comparing the expression levels quantified at step i) with their predetermined references values and
(iii) Administering to said subject a therapeutically effective amount of a radiotherapeutic agent and/or an immunotherapeutic agent and/or a chemotherapeutic agent and/or the methylation level of CACNA1C-AS1.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, the treatment consists of administering to the subject a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In some embodiments, the treatment consists of administering to the subject a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In some embodiment, the radiotherapy consists of proton therapy or proton radiotherapy. The term “proton therapy” or “proton radiotherapy” refers to a type of particle therapy that uses a beam of protons to irradiate diseased tissue, most often in the treatment of cancer. The chief advantage of proton therapy over other types of external beam radiotherapy is that as a charged particle the dose is deposited over a narrow range of depth, and there is minimal entry, exit, or scattered radiation dose.

In some embodiments, the treatment consists of administering to the subject a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the treatment consists of administering to the subject an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies (Nivolumab for example), anti-PDL1 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

In some embodiments, the immunotherapeutic agent is an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, 3-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-6thyl]amino}-1,2,5-oxadiazole-3 carboximidamide:

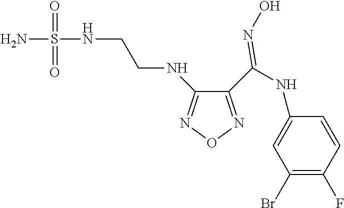 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

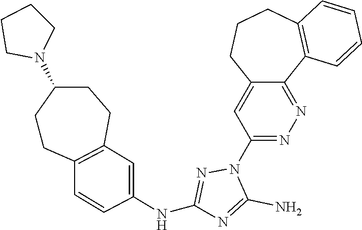 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “combination” is intended to refer to all forms of administration that provide a first drug together with a further (second, third . . . ) drug. The drugs may be administered simultaneous, separate or sequential and in any order. According to the invention, the drug is administered to the subject using any suitable method that enables the drug to reach the lungs. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the lungs.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the invention relates to a i) radiotherapeutic agent and ii) an immunotherapeutic agent for simultaneous, separate or sequential use in the treatment of a solid cancer.

In a particular embodiment, the invention relates to a i) chemotherapeutic agent and ii) an immunotherapeutic agent for simultaneous, separate or sequential use in the treatment of a solid cancer.

In a particular embodiment, the invention relates to a i) radiotherapeutic agent and ii) a chemotherapeutic agent for simultaneous, separate or sequential use in the treatment of a solid cancer.

In some embodiments, the subject will be treated with a radiotherapeutic agent in combination with an immunotherapeutic agent if the expression level of CACNA1C is higher than its predetermined reference value.

In some embodiments, the subject will be treated with a radiotherapeutic agent in combination with a chemotherapeutic agent if the expression level of CACNA1C is higher than its predetermined reference value.

In some embodiments, the subject will be treated with an immunotherapeutic agent in combination with a chemotherapeutic agent if the expression level of CACNA1C is higher than its predetermined reference value.

In some embodiments, the subject will be treated with a radiotherapeutic agent in combination with an immunotherapeutic agent if the expression level of CACNA1C is higher than its predetermined reference value, if the expression levels of CACNA1C-IT3, NDRG1, HBA2, HBB, IGLL5, ALM3, PTP4A3, BTG2, PAXX, KIAA0040, FLOT1, DPP7, HBA1, HYI, VIM, GNAS are higher than their predetermined reference value and if the expression levels of UTP20, NUP93, GRIK2, PPP3CC, LRIG3, CSMD1, ATP7B, EIF2AK3, NIPAL2, CROT, NIPAL3, CCT6B, ALG9, TMTC3, TRIP12 are lower than their predetermined reference value.

In some embodiments, the subject will be treated with a radiotherapeutic agent in combination with a chemotherapeutic agent if the expression level of CACNA1C is higher than its predetermined reference value, if the expression levels of CACNA1C-IT3, NDRG1, HBA2, HBB, IGLL5, ALM3, PTP4A3, BTG2, PAXX, KIAA0040, FLOT1, DPP7, HBA1, HYI, VIM, GNAS are higher than their predetermined reference value and if the expression levels of UTP20, NUP93, GRIK2, PPP3CC, LRIG3, CSMD1, ATP7B, EIF2AK3, NIPAL2, CROT, NIPAL3, CCT6B, ALG9, TMTC3, TRIP12 are lower than their predetermined reference value.

In some embodiments, the subject will be treated with an immunotherapeutic agent in combination with a chemotherapeutic agent if the expression level of CACNA1C is higher than its predetermined reference value, if the expression level of CACNA1C-IT3, NDRG1, HBA2, HBB, IGLL5, ALM3, PTP4A3, BTG2, PAXX, KIAA0040, FLOT1, DPP7, HBA1, HYI, VIM, GNAS are higher than their predetermined reference value and if the expression levels of UTP20, NUP93, GRIK2, PPP3CC, LRIG3, CSMD1, ATP7B, EIF2AK3, NIPAL2, CROT, NIPAL3, CCT6B, ALG9, TMTC3, TRIP12 are lower than their predetermined reference value.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the radiotherapeutic agent, the immunotherapeutic agent and/or the chemotherapeutic agent as described above are administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The compositions used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Analyses of Methylome data. R values of the CpG probes on the CACNA1C/CACNA1C-AS1 locus in cancerous and non-cancerous nasal cavity samples. The horizontal line corresponding to a p value of 0.2 delimits the threshold between unmethylated status and hemimethylated status for each CpG probe.

FIG. 2. Exemplary genes expression. CACNA1C (A), CACNA1C-AS1 (B), CACNA1C-IT3 (C) and CDX2 (D) mean expression in the NECT groups. Black star means profile significantly different from Normal samples with correction for multitesting.

FIG. 3. Predictor. Calculation of Pearson's correlation to a CT mean profile for each sample.

### EXAMPLE

### Material & Methods

### Ethical Approval

The present study included patients following strict human subjects protection guidelines, informed consent and IRB (Institutional Review Board) review of protocols in accordance with in accordance with The Code of Ethics of the World Medical Association (Declaration of Helsinki). All patients provided written informed consent prior to their inclusion. This trial was registered at NIH ClinicalTrials.gov (https://www.clinicaltrials.gov/), number NCT0281823.

### General Design and Population

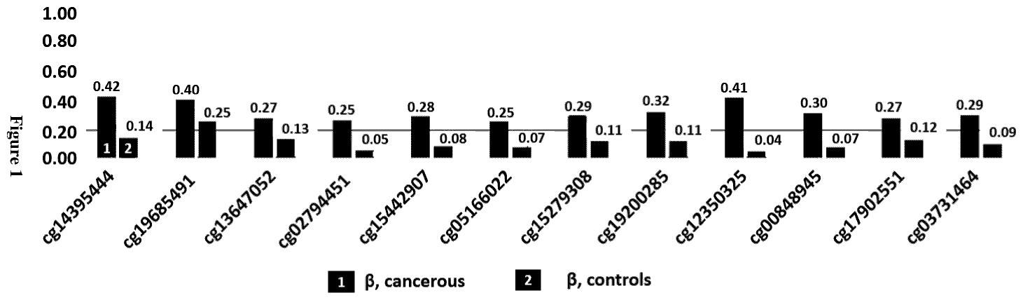

A first set of woodworkers (group 1) with unilateral nasal intestinal adenocarcinomas, operated on during a three-year period (2014-2016) were included in this study. Samples were collected in olfactory clefts by a non-invasive swabbing technique (for transcriptome analysis), and by biopsies (for DNA methylome and western blot analysis). Then we included wood dust exposed volunteers (group 2) and healthy non exposed volunteers (group 3) as controls, during a 3-months period (June 2016-August 2016) and retrieved samples from the most accessible olfactory cleft by the same brushing technique (cf. general design, FIG. 1). For ethical reasons, we were not allowed to take biopsies on healthy subjects, therefore we used a previously published methylation dataset of olfactory clefts of asthmatic patients (without wood dust exposure) as controls for methylome analysis [dataset] Yang I V et al., The Nasal Methylome and Childhood Atopic Asthma, Gene Expression Omnibus, 2015, https://www.ncbi.nlm.nih.gov/geo/query/acc.cgi?acc=GSE65163, GSE65163 [Yang IV, 2014].

We firstly compared tumoral samples (T and T′ samples) with healthy non-exposed volunteers' samples (N and N′ samples), both in transcriptome and in methylome analysis. We also investigated wood dust induced transcriptome modifications of exposed (without tumor) woodworkers' samples (E) and of contralateral sides (C samples) of woodworkers with tumors. The initial global transcriptomic analysis was then refined by a specific microarray data analysis focused on the genes highlighted in methylome analysis.

In order to limit biases due to sex, age, genetic abnormalities or inflammatory disease (unrelated to wood dust exposure), the criteria for non-inclusion were as follows for all subjects: 1/age inferior to 50 years 2/previous history of nasal irradiation 3/chronic inflammatory disease affecting the nasal cavity (nasal polyposis, systemic disease, cystic fibrosis), 4/any genetic disease known to be a risk factor for cancer (xeroderma pigmentosum, chromosomal aberrations, abnormalities in DNA repair).

### Brushing Technique and Samples Collection

Local anesthesia was performed using 1% Xylocaine spray. The brush (bronchial cytologic brush Asept Inmed diameter 1.8 mm/length 1200 mm) was inserted into the nasal cavity, within a curved sucker to guide the introduction into the olfactory cleft. The brush was moved forward in the olfactory cleft, until its tip was in contact with the anterior wall of the sphenoid, then moved back from 2 cm, still sheathed. Then the brush was pushed forward outside of its sheat, and moved 3 times in a back and forth motion of 2 cm length. When brushing was complete, the brush was withdrawn into the sheath and retracted through the curved sucker. The brush was cut and placed in 0.5 mL of RNAlater medium. The samples were sent to the pathology laboratory, in the same conditions than frozen sections, to be immediately vortexed and frozen (tube vertically placed in liquid nitrogen, then stored at −80° C.). For woodworkers with tumor, biopsies were additionally taken in both olfactory clefts, at the same level, and sent to the pathology laboratory in the same conditions.

All subjects were asked to rate 1/perceived usefulness of the screening method, 2/acceptability, 3/pain. They were also asked if they would accept to be screened again by the same method.

### Purification of RNA

Briefly, samples were incubated with 50 μL of TRIzol (Invitrogen, Carlsbad, CA) reagent at room temperature for 15 minutes to extract DNA. Two methods were used: with cellular suspension only (after centrifugation) or with cellular suspension and brushes. Samples were vortexed. Phase separation was ensured by adding 200 μL of chloroform. Samples were centrifuged at 10,000 g for 10 min and glycogen was added as a carrier. Then we added 0.5 mL of isopropanol per each milliliter of the clear phase. After mixing and precipitation (10 min), we collected the precipitated RNA by centrifugation at 10,000g in a centrifuge for 10 min at 4° C. After decantation of the supernatant, we removed remaining liquid and immediately resuspended the pellet (without drying) in 1×SDS solubilization buffer. We added NaOAc to 3 M, re-extracted the solution with phenol and ensured precipitation with ethanol.

### Transcriptome Analysis

RNA quantities were determined using the Nanodrop Spectrophotometer ND-1000 and the Qubit™ 2.0 Fluorometer with the Qubit™ RNA HR Assay Kit. We assessed RNA integrity using RNA 6000 PicoChips with Agilent 2100 BioAnalyzer. All RNA samples had a low RIN number <6. Transcriptome profiling was conducted using Affymetrix Genechip Human Gene 2.0 ST Array (Thermofisher) following GeneChip WT Pico Reagent Kit Manual Target Preparation for GeneChip® Whole Transcript (WT) Expression Arrays UserGuide P/N 703262 Rev. 2

### Microarray Data Analyses

Analyses were performed as in Chen et al. 2015 and Baron et al. 2011 [Chen, 2015; Baron, 2011]. Scanned signals were quantified from all microarrays by GenePix Pro software version 5.1 (Axon Instruments, Union City, CA). Signals were normalized against a median profile using Lowess method [Yang et al. 2002]. Genes with similar profiles were grouped using k-means (k=10 and 100 runs) algorithm [De Hoon, 2004] on log transformed and median center genes with Pearson coefficient as distance and average linkage clustering method. Data were visualized using JavaTreeView [Saldanha, 2004].

Gene signatures were functionally annotated with GoMiner [Zeeberg, 2003] and Gene Ontology [Ashburner, 2000]. The specific microarray data analysis was focused on CDX2 and its main downstream genes and on genes identified by DNA methylome analysis (CACNA1C and SLC26A10).

We built a gene predictor to predict nasal intestinal adenocarcinoma existence in patients, by selecting the best genes separating N/E from C/T samples. To achieve this goal, we selected the Top-100 genes from cluster 1 and the Top-100 genes from cluster 3. We calculated a mean CT profile and calculated Pearson's correlation with this profile for each sample. Separation quality was estimated by Fisher Exact Test, Sensitivity by calculating True positive rate and Specificity by calculating True negative rate. Samples giving a weak correlation (between −0.25 and 0.25) were considered as unpredictable.

### DNA Methylome Analyses

We carried out bisulfite conversion of 600 ng of tumor tissue DNA using EZ DNA Methylation kit (Zymo Research, Proteigene, Saint-Marcel, France). The genome-wide profiling of DNA methylome was determined using the Infinium MethylationEPIC BeadChip array (Illumina, Paris, France), according to the manufacturer's instructions. The Infinium MethylationEPIC BeadChip provides coverage of 850,000 CpG probes in enhancer regions, gene bodies, promoters, and CpG islands. The arrays were scanned on an Illumina iScan® system, and raw methylation data were extracted using Illumina's Genome Studio methylation module. For each CpG probe, the methylation level was described as a p value, ranging between 0 (fully unmethylated CpG probe) and 1 (fully methylated CpG probe). Background correction and normalization was implemented using SWAN method (R Package Minfi) [Aryee, 2014]. We visually inspected the whole-genome distribution of the CpG probes according to their R value. Samples that did not show a methylation profile consistent with a beta distribution were excluded from the epigenome-wise association study (EWAS) and were used in the secondary locus-specific analysis. In the EWAS, we compared the whole DNA methylome profile of nasal cavity cancerous samples with that of 72 non-cancerous nasal cavity methylomes profiles retrieved from the GEO database (GSE65163) [Yang, 2017]. For each CpG probe, we compared the mean R values between cancerous and non-cancerous samples using t-test with Bonferroni correction to account for the multiple testing issue. Due to the low sample size, and considering the exploratory approach of our analysis, we used the smoothed P-value transformation by converting nominal P-values obtained from the t-test to smoothed P-values using a window radius of 3, as previously reported [Gueant J L, 2018]. All statistical analyses were performed using the SNP & Variation Suite (v8.8.1; Golden Helix, Inc., Bozeman, MT, USA).

### Immunochemistry

To confirm the observed transcriptomic and methylome modifications, tumor sections were reacted with specific antibodies (CDX2 (clone DAK-CDX2, 1:100 dilution, DAKO, Carpinteria, CA, USA), CACNA1C (HPA039796, 1:50 dilution, Sigma-Aldrich, St Louis, MO, USA), SLC26A10 (primary antibody: HPA044719, 1:200 dilution, Sigma-Aldrich, St Louis, MO, USA), then were stained immunohistochemically by the avidinbiotin complex method. Tumoral tissue stainings were compared to non tumoral adjacent mucosa stainings as controls.

### Data Availability

Data are available at Gene Expression Omnibus under the GSEx reference for transcriptome data and GSEy for methylation data.

### Results

### Population

Sixteen woodworkers with unilateral intestinal type sinonasal adenocarcinomas (group 1), 16 woodworkers without cancer (group 2) and 13 healthy subjects (group 3) were included. Exposure to wood dust and main other carcinogens is presented in Table I. Woodworkers with adenocarcinomas were slightly older than controls. The latency was longer in group 1 (adenocarcinoma) than in group 2, while exposure duration was longer in group 2. There were no other difference between groups. In particular, we could not identify any differences between group 1 and 2 in the wood types processed or in woodworkers activities.

### Brushing Technique and Samples Collection

### Brushing Technique Evaluation

The brushing was relatively easy to perform and well tolerated: there were no side effects and it was usually painless (3 volunteers refused local anesthesia, mean numerical rating scale: 2.7±1.8/10, maximum: 8 in a woodworker with a hudge septal deviation). The sampling method was found to have an excellent «acceptability», and «perceived usefulness» (9.1±1.4/10 and 8.4±1.4/10 respectively) and all volunteers «would accept to do it again if necessary».

### Samples

In two woodworkers, tumoral side samples (T and T′) were not included for analysis, because a prior recent but incomplete tumoral exeresis might have influenced the results on this side.

### Global Microarray Data Analysis

A global transcriptome analysis was performed to find out if it was possible to distinguish the different samples profiles. We identified 4 differential clusters between T and N samples. No differential cluster could be identified between N and E samples. Finally, two clusters could identify healthy subjects samples (E and N) and samples of woodworkers with a tumor (T) even in contralateral sides samples (C). These differences persisted even when adjusting for potential confounders (age or wood dust exposure duration and/or latency).

We then search for functional annotation enrichment of these clusters (Data not shown).

Cluster 1, overexpressed in C and T, is enriched in genes involved in extracellular matrix such as collagens, matrix metalloproteinases (MMP8, MMP9, MMP11, MMP19, MMP20), leukocytes differentiation (Runxl, GATA1, GATA3, NFKBID, RELB and interleukins), and muscle contraction such as myosin chains. This suggests a change in cellularity (increase in contractile cells and leukocytes) of CT samples.

Cluster 3, underexpressed in C and T samples, is enriched in nuclear pore genes such as nucleoporins (NUP37, NUP43, NUP85, NUP98, NUP107, NUP133, NUP160 . . . ) and preassembly of GPI anchor in ER membrane genes such as Phosphatidylinositol glycan anchor genes (PIGB, PIGC, PIGF, PIGH, PIGL, PIGM, PIGN, PIGO, PIGV, PIGW . . . ). This suggests a decrease in protein transport in CT samples.

Cluster 4, overexpressed in T samples, is enriched in genes involved in DNA replication such as DNA replication complex (GINS1, GINS2, MCM3, MCM4, MCM6, MCM7, MCM10, CDC6, CDC45, CDC47, ORC1, ORC6 . . . ), and in laminin binding such as integrins (ECM1, ITGA6, ITGA7, ITGA9, ITGB1, THBS1, LGALS1 . . . ). These genes are involved in cell proliferation and cancers. In particular ECM1 is implicated in breast cancer, thyroid cancer, hepatocellular carcinoma, and other cancers, and also in ulcerative colitis. Galectin-1 (LGALS1) may act as an autocrine negative growth factor that regulates cell proliferation and is involved in Hodgkin Lymphoma. Cluster 4 also contain two exemplary genes: CDX2 involved in ITACs and the calcium channel, voltage-dependent, L type, alpha 1C subunit (CACNAC). This suggests increase in cell division in T samples.

Cluster 5 underexpressed in T samples, is strongly enriched in genes involved in cilium functions: axonemal complex (CCDC39, CCDC40, CCDC103, CCDC114, DNAAF1, DNAAF3, DNAH10, DNAH11, DNAI2, DNALI1 . . . ) and ciliary cell motility (BBS1, BBS4, DAW1, IFT46, RFX3 . . . ). Diminution of cluster 5 gene expression in tumors probably reflects the change in cellularity: diminution of ciliary cells due to invasion of tumoral epithelial cells.

### DNA Methylome Analysis

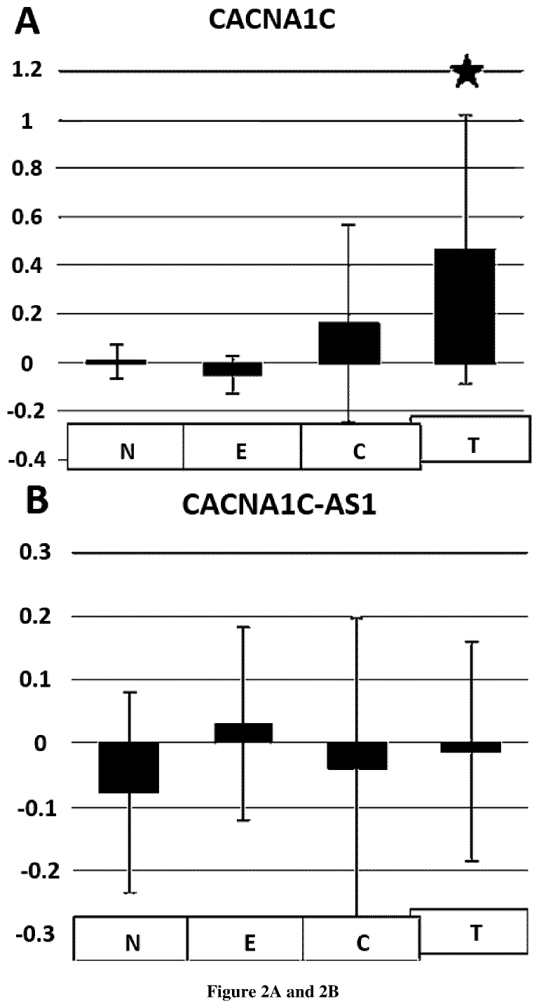

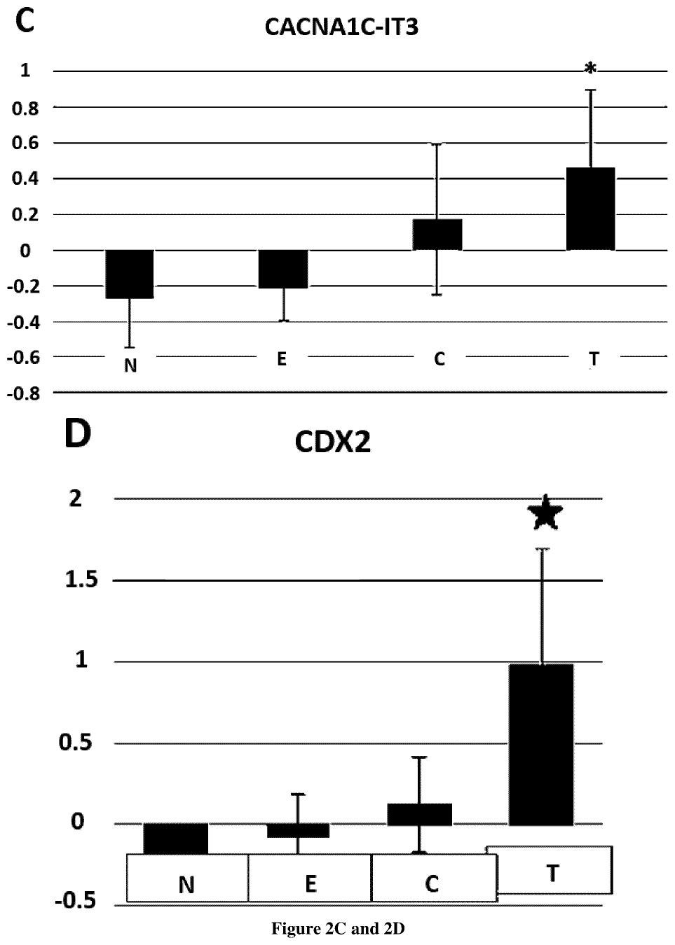

Of the fourteen tumoral samples, eleven could be analyzed for their DNA methylation profile using the Infinium Methylation EPIC array. Among them, four samples exhibited high-quality metrics and were included in the EWAS phase of the study. The remaining samples (n=7) exhibited sub-optimal quality metrics and were included in the secondary locus-specific analysis of the study which focused on the top loci retrieved in the EWAS phase. In the EWAS, we retrieved two top loci, namely: CACNA1C/CACNA1C-AS1 and SLC26A10. The first top locus corresponded to the CpG island ‘CpG: 84’ which locates in the promoter 5′ region of the CACNA1C Antisense RNA 1 (CACNA1C-AS1) and the 3′UTR region of the CACNA1C gene. The CpG probes reported in this locus exhibited a hemimethylated profile (P values between 0.2 and 0.6) among cancerous samples and a fully unmethylated profile among non-cancerous samples (FIG. 1). The second top locus retrieved in the EWAS corresponded to the CpG island ‘CpG:41’ which locates in the promoter region of the SLC26A10 gene. This locus encompasses nine CpG probes that exhibited a hemimethylated profile among cancerous samples and a fully unmethylated profile among non-cancerous samples ( ). In the secondary locus-specific analysis, the assessment of methylation profiles of the seven cancerous samples with a suboptimal quality metrics confirmed the overall hemimethylated profile of the two top loci CACNAC/CACNA1C-AS1 and SLC26A10 (supplementary Tables II and III). The visual inspection of the volcano plot confirmed the clustering of the CpG probes belonging to the CACNA1C/CACNA1C-AS1 (FIGS. 2A and 2B) and SLC26A10 loci (Data not shown). The DNA methylation profiles of cancerous and non-cancerous cell lines did not differ significantly regarding the CDX2 locus (supplementary Table III).

### Specific Microarray Data Analysis

We looked for expression of CDX2 and of differentially methylated genes: CACNA1C, CACNA1C-AS, CACNA1C-AS1 (FIGS. 2A, 2B and 2C) and SLC26A10 (Data not shown). CDX2 expression was significantly increased in T samples (Fold change of 2.46 for T/N samples), but also in some C samples (FIG. 2D). CACNA1C-AS and SLC26A10 expressions were weakly modified but a significant effect could be observed on CACNA1C with a fold change of 1.52.

### Immunochemistry

A strong nuclear staining for CDX2 was confirmed in all tumoral samples (Data not shown). CACNA1C immunostaining was focally positive in tumoral samples (Data not shown) while negative in adjacent non tumoral mucosa. SLC26A10 was strongly positive in both tissues (Data not shown).

### ITAC Predictor

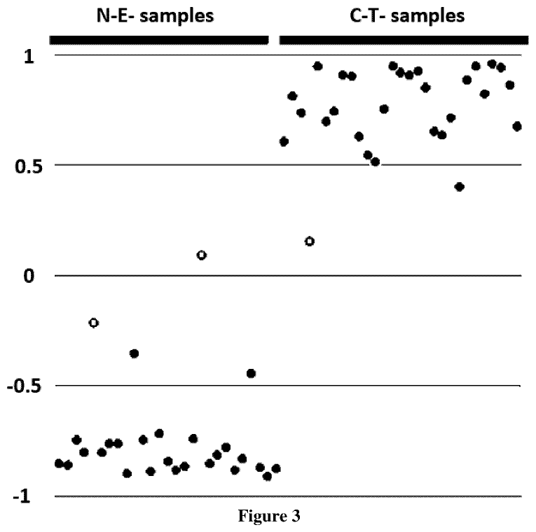

Hierarchical classification of genes and samples showed a clear separation between NE and CT samples (Data not shown). A C/T mean profile was created and Pearson's correlation with this profile was calculated for each sample (FIG. 3). Weak correlations were considered as unpredictable. This predictor was highly significant and had a sensibility of 97% and a specificity of 93% (Data not shown).

### DISCUSSION

With a non-invasive sampling technique, we identified several transcriptional modifications that might be useful in distinguishing the different categories of samples, in particular tumoral samples, through their specific transcriptomic profile.

The main changes in tumoral cells transcriptome (when compared to healthy mucosa cells) were consistent with carcinogenesis and tumor phenotype: we mainly observed a decreased expression in the ‘cilium’ cluster and an increased expression in the ‘DNA replication initiation’ cluster. Indeed, most of nasal wood dust induced adenocarcinomas exhibit an intestinal phenotype [Gallet, 2018], although nasal mucosa is normally constituted by a respiratory pseudo-stratified epithelium with ciliary cells and sero-mucous glands. Consistently with its presumed key role in ITACs cancerogenesis [De Gabory, 2008; Gallet, 2018; Kennedy, 2004], CDX2 was overexpressed in all tumoral samples. The mechanisms and the signification of this overexpression remain obscure. While CDX2 overexpression has also been described in various non intestinal nasal carcinomas, CDX2 overexpression seems to be mandatory for the acquisition of an intestinal type phenotype. Interestingly, these woodworkers also exhibited increased CDX2 expression in their contralateral epithelium, while there was no tumor. This is consistent with previous findings [Barros, 2012]: CDX2 overexpression alone seems unsufficient for tumorogenesis, but might still be an early key event enabling the further development of a malignant tumor. From this perspective, the analogy between Barrett's adenocarcinomas and ITACs [Gallet, 2018] is interesting because both mucosa share a same embryologic origin and the same transdifferenciation towards an intestinal type phenotype when exposed to a specific carcinogen. As biliar acids in Barrett's adenocarcinoma, chronic wood dust exposure also induces chronic inflammation in woodworkers nasal fossa (Bucci, 2002). In Barrett's adenocarcinoma, the role of CDX2 is linked to inflammation regulation (COX2, PPAR, NFKB) [Villanacci, 2007] and inflammation induced CDX2 promoter methylation might orient differentiation towards an intestinal or squamous cell carcinoma phenotype [Wu, 2013; Guo, 2007]. However, in our study CDX2 expression was not associated with methylation changes. Additionally, wood dust induced chronic nasal inflammation was not associated with major transcriptomic changes: the transcriptomic profile of healthy (without tumor) exposed woodworkers' mucosa was not different from that of non-exposed controls.

A contrario, woodworkers with ITAC exhibited a different transcriptomic profile both in their tumoral and contralateral epithelium, when compared to wood dust exposed subjects and non exposed subjects. When comparing these samples with controls, two clusters were highly differential. The first one mainly consisted in an overexpression in collagen or extracellular matrix catabolism and regulation of leukocytes differentiation. This might be the witness of a specific inflammation. The second one consisted in a decrease of expression in membrane and nuclear pore activity. The signification of these modifications is unclear. In a prior transcriptomic analysis of gene expression profiling of 26 nasal adenocarcinomas, Tripodi [Tripodi, 2008] identified three genes differentially expressed (LGALS4 (regulating cell-cell and cell-matrix interactions), CLU (a stress protein) and ACS5 (fatty acids metabolism)). None of these was confirmed in our study, but it should be emphasized that Tripodi included many non intestinal adenocarcinomas, which might exhibit a very different transcriptomic profile. Altogether, our results would suggest that these woodworkers who developed a tumor might have had a specific wood dust sensitivity or that they had experimented a different type of exposure. Indeed, in 4 patients, the contralateral tumor was so small that it is unlikely that it could have influenced the contralateral sides transcriptomic profiles in any way. The slight differences in exposure latency or in woodworkers' age between groups do not explain this result either, as transcriptomic profiles were still different when adjusting for counfounders. In light of these bilateral modifications, the rarity of bilateral ITACs might appear surprising, but it could be due to the long latency required before developing any tumoral lesion.

There are few data on methylation in ITAC in the literature: for Perrone (Perrone, 2003), p14(ARF) and p16(INK4a) were frequently hypermethylated, while for Perez-Ordonez [Perez-Ordonez, 2008], promoter methylation of MMR genes do not play a role in the pathogenesis of ITAC. In methylome analysis, we identified two sites of interest: CACNA1C-AS and SLC26A10.

CACNA1C gene encodes an alpha-1 subunit of a voltage-dependent calcium channel. It shares superpathways mainly with TCR signaling, circadian entrainment, aldosterone synthesis and secretion. Until now, CACNA1C modifications have been mainly described in autism, epilepsy, atrial fibrillation and bronchitis. Mutations of CACNA1C are involved in cardiomyopathies such as Long QT and Brugada syndromes [Béziau, 2014]. But CACNA1C overexpression has already been described in colorectal or gastric adenocarcinomas [Wang, 2015] with CACNA1C appearing in the top 10% of the most augmented genes. Its role is still unknown, but Calcium channels play a crucial role in cell proliferation, migration and apoptosis and might therefore contribute to cancer development [Wang, 2015]. In our study, the hemi-methylation of the CPG codon observed should result in haploinsufficiency on antisense, that effectively results in an increase in CACNA1 expression, confirmed in immunochemistry. These alterations both in methylome and in transcriptome underline the particular role of CACNA1C:; it may be considered as a Master Gene of ITAC [Chen, 2015].

SLC26 gene family encodes transmembrane solute carriers: SLC26A10 over-expression has been described in neuroblastoma or glioma, while its under-expression has been described in gastric carcinoma [Alper, 2013]. In this study, significant SLC26A10 associations with levels of eicosapentanoic and arachidonic acids have been described, but the link with ITACs and nasal inflammation is not clear [Alper, 2013]. Though, in our case, hemimethylation of SLC26A10 promoter do not seem to decrease SLC26A10 expression in a significant manner, as it appears naturally highly expressed in olfactory cleft mucosa.

Prediction of ITAC samples allows to distinguish NE samples from CT samples with 100% of sensibility and 100% of specificity if unpredictable samples are not taken in account. This together with our non-invasive sampling method raises the possibility of an easy diagnostic process to detect ITAC. This demonstrate that integrative genomics approach allows prediction of Tumour and Contralateral samples and underline the role CACNA1 as a Master Gene of ITAC.

### CONCLUSION

Thanks to a non-invasive brushing technique, it has been possible to identify transcriptomic and methylation modifications that are consistent with phenotypic profiles and ITACs natural history, thus making it possible to identify subjects with an ITAC. Our study revealed differences that have not yet been described for CACNA1C-AS and SLC26A10. In particular, the study shows that CACNA1C can be used as a biomarker of ITAC. Our results paves the way for a simple and non-invasive diagnosis method for ITAC.

### REFERENCES

Throughout this application, various references describe the state of the art to which this invention pertains. The disclosures of these references are hereby incorporated by reference into the present disclosure.
Alper S L, Sharma A K. The SLC26 gene family of anion transporters and channels. Mol Aspects Med. 2013 April-June; 34(2-3):494-515. doi: 10.1016/j.mam.2012.07.009.
Ashburner M, Ball C A, Blake J A, Botstein D, Butler H, Cherry J M et al., Gene ontology: tool for the unification of biology. The Gene Ontology Consortium. Nat. Genet. 2000, 25, 25-29.
Aryee M J, Jaffe A E, Corrada-Bravo H, Ladd-Acosta C, Feinberg A P, Hansen K D et al. Minfi: a flexible and comprehensive Bioconductor package for the analysis of Infinium DNA methylation microarrays. Bioinformatics 2014; 30: 1363-9.#
Baron D, Dubois E, Bihouee A, Teusan R, Steenman M, Jourdon P et al., Metaanalysis of muscle transcriptome data using the MADMuscle database reveals biologically relevant gene patterns. BMC Genomics 2011, 12, 113-126.
Barnes L. Intestinal-type adenocarcinoma of the nasal cavity and paranasal sinuses. Am J Surg Pathol. 1986; 10:192-202.
Barros R, Freund I N, David L, Almeida R. Gastric intestinal metaplasia revisited: function and regulation of CDX2. Trends Mol Med. 2012 September; 18(9):555-63. doi: 10.1016/j.molmed.2012.07.006. Epub 2012 Aug. 4.
Béziau D M, Barc J, O'Hara T, Le Gloan L, Amarouch M Y, Solnon A et al. Complex Brugada syndrome inheritance in a family harbouring compound SCN5A and CACNA1C mutations. Basic Res Cardiol. 2014; 109(6):446-460
Bonato M, Piantanida R, Riva C, Cis C, Capella C. [Intestinal type adenocarcinoma of the nose and paranasal sinuses. Histological and immunohistochemical study of 14 cases]. Acta Otorhinolaryngol Ital. Juin 1989; 9(3):297 310.
Carton M, Goldberg M, Luce D. [Occupational exposure to wood dust. Health effects and exposure limit values]. Rev Epidemiol Sante Publique. Avr 2002; 50(2):159 178
Chen G, Broséus J, Hergalant S, Donnart A, Chevalier C, Bolanos-Jimenez F et al. Mol. Nutr. Food Res. 2015, 59, 293-302
Choi H-R, Sturgis E M, Rashid A, DeMonte F, Luna M A, Batsakis J G, et al. Sinonasal adenocarcinoma: evidence for histogenetic divergence of the enteric and nonenteric phenotypes. Hum. Pathol. nov 2003; 34(11):1101 1107.
Choussy O, Ferron C, Vedrine P-O, Toussaint B, Lietin B, Marandas P, et al. Adenocarcinoma of Ethmoid: a GETTEC retrospective multicenter study of 418 cases. Laryngoscope. mars 2008; 118(3):437 443.
De Gabory L, Conso F, Barry B, Stoll D. [Carcinogenesis of the ethmoidal adenocarcinoma due to wood dust]. Rev Laryngol Otol Rhinol (Bord). 2009; 130(2):93 104.
De Hoon M J, Imoto S, Nolan J, Miyano S. Open source clustering software. Bioinformatics 2004, 20, 1453-1454.
Demers P A, Kogevinas M, Boffetta P, Leclerc A, Luce D, Gerin M, et al. Wood dust and sino-nasal cancer: pooled reanalysis of twelve case-control studies. Am. J. Ind. Med. August 1995; 28(2):151 166.
Donhuijsen K, Hattenberger S, Schroeder H G. [Nasal sinus carcinoma after wood dust exposure. Morphological spectrum of 100 cases]. Pathologe. 2004 February; 25(1):14-20.
Gallet P, Nguyen D T, Russel A, Jankowski R, Vigouroux C, Rumeau C. Intestinal and non-intestinal nasal cavity adenocarcinoma: Impact of wood dust exposure. Eur Ann Otorhinolaryngol Head Neck Dis. 2018 December; 135(6):383-387. doi: 10.1016/j.anorl.2018.08.012. Epub 2018 Sep. 7.
Georgel T, Jankowski R, Henrot P, Baumann C, Kacha S, Grignon B, et al. C T assessment of woodworkers' nasal adenocarcinomas confirms the origin in the olfactory cleft. AJNR Am J Neuroradiol. août 2009; 30(7):1440 1444.
Gueant J L, Chery C, Oussalah A, Nadaf J, Coelho D, Josse T et al. APRDX1 mutant allele causes a MMACHC secondary epimutation in cblC patients. Nat Commun 2018; 9: 67.
Guo M, House M G, Suzuki H, Ye Y, Brock M V, Lu F et al. Epigenetic silencing of CDX2 is a feature of squamous esophageal cancer. Int J Cancer. 2007 Sep. 15; 121(6):1219-26.
Jankowski R, Georgel T, Vignaud J M, Hemmaoui B, Toussaint B, Graff P, et al. Endoscopic surgery reveals that woodworkers' adenocarcinomas originate in the olfactory cleft. Rhinology. déc 2007; 45(4):308 314.
Kennedy M T, Jordan R C, Berean K W, Perez-Ordonez B. Expression pattern of CK7, CK20, CDX-2, and villin in intestinal-type sinonasal adenocarcinoma. J Clin Pathol. 2004 September; 57(9):932-7.
Perrone F, Oggionni M, Birindelli S, Suardi S, Tabano S, Romano R, et al. «TP53, p14ARF, p161NK4a and H-ras gene molecular analysis in intestinal-type adenocarcinoma of the nasal cavity and paranasal sinuses». International Journal of Cancer. Journal International Du Cancer 105, no. 2 (juin 10, 2003): 196-203.
Perez-Ordonez B, Huynh N N, Berean K W, Jordan R C. Expression of mismatch repair proteins, beta catenin, and E cadherin in intestinal-type sinonasal adenocarcinoma. J Clin Pathol. 2004 October; 57(10):1080-3.
Saldanha A J, Java Treeview-extensible visualization of microarray data. Bioinformatics 2004, 20, 3246-3248.
Tripodi D, Quéméner S, Renaudin K, Ferron C, Malard O, Guisle-Marsollier I et al. Gene expression profiling in sinonasal adenocarcinoma. BMC Med Genomics. 2009 Nov. 10; 2:65. doi: 10.1186/1755-8794-2-65.
Villanacci V, Rossi E, Zambelli C, Galletti A, Cestari R, Missale G, Casa D D, Bassotti G. COX-2, CDX2, and CDC2 immunohistochemical assessment for dysplasia-carcinoma progression in Barrett's esophagus. Dig Liver Dis. 2007 April; 39(4):305-11.
Vivanco B, Llorente J L, Perez-Escuredo J, Alvarez Marcos C, Fresno M F, Hermsen M A. Benign lesions in mucosa adjacent to intestinal-type sinonasal adenocarcinoma. Patholog Res Int. 2011 Apr. 20; 2011:230147. doi: 10.4061/2011/230147.
Wang C Y, Lai M D, Phan N N, Sun Z, Lin Y C. Meta-Analysis of Public Microarray Datasets Reveals Voltage-Gated Calcium Gene Signatures in Clinical Cancer Patients. PLoS One. 2015 Jul. 6; 10(7):e0125766.
Wilhelmsson B, Lundh B, Drettner B, Stenkvist B. Effects of wood dust exposure and diethylnitrosamine. A pilot study in Syrian golden hamsters. Acta Otolaryngol. Févr 1985; 99(1-2):160 171.
Wu W, Bhagat T D, Yang X, Song J H, Cheng Y, Agarwal R et al. Hypomethylation of noncoding DNA regions and overexpression of the long noncoding RNA, AFAP1-AS1, in Barrett's esophagus and esophageal adenocarcinoma. Gastroenterology. 2013 May; 144(5):956-966.e4
Yang I V, Pedersen B S, Liu A H, O'Connor G T, Pillai D, Kattan M et al. The nasal methylome and childhood atopic asthma. J Allergy Clin Immunol 2017; 139: 1478-88.
Yang Y H, Dudoit S, Luu P, Lin D M, Peng V, Ngai J et al., Normalization for cDNA microarray data: a robust composite method addressing single and multiple slide systematic variation. Nucleic Acids Res. 2002, 30, e15.
Zeeberg B R, Feng W, Wang G, Wang M D, Fojo A T, Sunshine M et al., GoMiner: a resource for biological interpretation of genomic and proteomic data. Genome Biol. 2003, 4, R28.

1. A method of determining whether a subject has or is at risk of having nasal intestinal type adenocarcinoma (ITAC) and treating the subject comprising,
determining the expression level of CACNA1C in a sample from the subject, and
administering to the subject a therapeutically effective amount of chemotherapy, radiotherapy, and/or immunotherapy when said expression level is higher than a control expression level established in subjects who do not have ITAC.

2. The method according to claim 1 wherein said method comprises determining the expression level of CACNA1C in combination with at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3, and TRIP12.

3. The method according to claim 1 wherein said method comprises determining the methylation level of CACNA1C-AS.

4. (canceled)

5. A method for treating a subject suffering from ITAC, wherein said method comprises the steps of:
(i) determining in a biological sample obtained from said subject the expression level of CACNA1C
(ii) comparing the expression level quantified at step i) with a predetermined reference value and
(iii) when the expression level is higher than the predetermined reference value administering to said subject a therapeutically effective amount of a radiotherapeutic agent and/or an immunotherapeutic agent and/or a chemotherapeutic agent.

6. The method according to claim 5 wherein said method further comprises the step of:
(i) determining a) the expression level of at least one gene selected from the group consisting of CACNA1C-IT3, NDRG1, HBA2, HBB, IGLL5, CALM3, PTP4A3, BTG2, PAXX, KIAA0040, FLOT1, DPP7, HBA1, HYI, VIM, GNAS, UTP20, NUP93, GRIK2, PPP3CC, LRIG3, CSMD1, ATP7B, EIF2AK3, NIPAL2, CROT, NIPAL3, CCT6B, ALG9, TMTC3 and/or TRIP12 and/or b) the methylation level of CACNA1C-AS1;
(ii) comparing the expression levels quantified at step i) with corresponding predetermined reference value and
(iii) when the expression levels are higher than the corresponding predetermined reference values, administering to said subject a therapeutically effective amount of a radiotherapeutic agent and/or an immunotherapeutic agent and/or a chemotherapeutic agent.

7. The method according to claim 1 wherein the subject is a woodworker.

8. The method according to claim 1 wherein the sample is collected in olfactory clefts.

9. The method according to claim 1 wherein the sample is collected with a brushing technique comprising insertion of a brush into the nasal cavity.
